(19) United States
(12) Patent Application Publication
(10) Pub. No.: US 2023/0389792 A1
(43) Pub. Date: Dec. 7, 2023

(54) SYSTEM AND METHOD OF CORNEAL SURFACE MEASUREMENT AUGMENTED FOR MACHINE INGESTION

(71) Applicant: Tilleron Inc, Great Neck, NY (US)

(72) Inventors: Joshua Abba Young, Great Neck, NY (US); Kathryn Masselam Hatch, Lexington, MA (US); Emma Nicole Young, Great Neck, NY (US)

(21) Appl. No.: 18/199,050

(22) Filed: May 18, 2023

Related U.S. Application Data
(60) Provisional application No. 63/349,085, filed on Jun. 4, 2022.

(51) Int. Cl. A61B 3/107; A61B 3/14; G06T 7/00

(52) U.S. Cl. CPC A61B 3/107; A61B 3/14; G06T 7/0012; H04N 23/56

(57) ABSTRACT

Methods, systems, and apparatus for illumination of the cornea of an eye are disclosed; in some implementations, such methods, systems, and apparatus may generally comprise or employ a light source and an optical mask, alignment of a viewing portal, and capture of a cornea-reflected image using a camera or other optical array. In some implementations, a system and method may generally comprise or involve generating data that are optimized for ingestion by an artificial intelligence or machine learning engine, thereby facilitating contact lens selection or detection of keratoconus or “dry eye” conditions for ophthalmic, optometric, and pediatric practices. Specifically, the disclosed subject matter teaches innovative cornea-reflective patterns, the reflections of which are designed for ingestion by statistical, machine learning, or artificial intelligence models.

### CROSS-REFERENCE TO RELATED APPLICATION(S)

This application claims the benefit of U.S. provisional patent application Ser. No. 63/349,085, filed Jun. 4, 2022, the disclosure of which is hereby incorporated herein by reference in its entirety.

### FIELD

Aspects of the disclosed subject matter relate generally to eye pathology detection and monitoring, and more particularly to a system and method of corneal surface measurement that enables capture of corneal surface information for automated analysis.

### BACKGROUND

Many pathologies affect the cornea. Some of these, like keratoconus, are treatable if the diagnosis is made while the patient remains young. Establishing these diagnoses has been the job of the trained clinician such as an ophthalmologist or an optometrist who has the requisite training and skill to make the appropriate determination. Tools employed by such clinicians include devices that measure the curvature of the surface of the cornea. These devices produce output that is interpretable by the clinician.

More recently, artificial intelligence and machine learning algorithms have been created that can aid in the interpretation of the display output that was originally designed for clinician interpretation. These algorithms suffer the disadvantage of interpreting displays or images that were originally created for human interpretation.

In some practical situations, by way of example, many cataract patients demonstrate evidence of a condition known as “dry eye” upon examination for cataract surgery. The majority of these patients are asymptomatic until testing. This ocular surface disease may result in inaccurate calculations for the power of intraocular lenses required, and may therefore result in a patient becoming unintentionally nearsighted or farsighted after cataract surgery. Remediation is possible, but only if this condition is detected before surgery.

By way of another example, in the case of contact lens fitting by optometrists, workflow inefficiencies exist that create undue expense and time commitments for both patients and care providers. Patients fit with contact lenses with which they are uncomfortable require increased time in office (e.g., known in the profession as “chair time”) with an optometrist, an ophthalmologist, or other care provider. As a result of discomfort, or frustration with conventional service paradigms, many contact lens wearers decline to continue wearing the devices within three years of agreeing to treatment. In some instances, it is believed that abnormalities of the tear film are associated with dissatisfaction experienced by contact lens wearers, but devices to measure tear quality (e.g., to quantify or otherwise to assess or to evaluate such tear film) over contact lenses are expensive and require substantial training, making determinations expensive and time consuming for both patient and care-giver.

By way of another example, keratoconus is a less common malady than the foregoing two conditions, but is equally identifiable and capable of being monitored by the subject matter disclosed below. Keratoconus is a progressive thinning and steepening of the cornea of a human eye, with a typical onset during the teenage years. New cross-linking therapy can stabilize the condition, but only in cases in which the diagnosis has been made. The chief problem is that although the age of onset is typically in a patient's teenage years, the mean age of diagnosis is a decade or more later, when patients present with more advanced disease than would be the case if the condition were detected earlier. Devices exist to detect keratoconus, but they are generally unavailable to pediatricians, and typically require substantial training in order to interpret the results that they generate.

There is, therefore, a need for an improved system and method employing data that have been captured in a manner designed for automated machine interpretation (e.g., a machine learning algorithm) which may then enable a device to render an interpretation of the acquired data. For example, it may be desirable in some instances to obtain (for subsequent processing) an image of a cornea or other body feature that is more advantageous for machine interpretation than it is for traditional interpretation methods that rely upon human intervention (and that only subsequently may be repurposed for machine ingestion).

### SUMMARY

The following presents a simplified summary of the disclosure in order to provide a basic understanding of some aspects of various embodiments disclosed herein. This summary is not an extensive overview of the disclosure. It is intended neither to identify key or critical elements of the disclosed embodiments nor to delineate the scope of those embodiments. Its sole purpose is to present some concepts of the disclosed subject matter in a simplified form as a prelude to the more detailed description that is presented later.

The present disclosure describes a system and method that are operative to gather data captured in a manner that is designed for automated machine interpretation. This feature may create an advantageous setting for a machine learning algorithm which may then render an interpretation of the data. Accordingly, an image may be created that is more advantageous for machine interpretation than traditional methods designed for human interpretation (where image data are later repurposed for use by machines or algorithms).

It will be appreciated that some advantages may include (but are not limited to) one or more of the following. By employing a light mask (an “optical mask” as described below) that incorporates many apices, an apex- and edge-rich corneal-reflected image may be captured. Circumferential displacement of these apices and edges is more evident than is available with traditional placido disk reflection techniques. In some implementations, artificial intelligence and machine learning algorithms may be employed to determine the probability of specific pathologies of the eye.

Implementations may include one or more of the following: illumination of the cornea of an eye using at least one light source oriented to pass light through an optical mask; alignment of a viewing portal to facilitate photography or other image capture of an image of the optical mask reflected from the cornea; and capture (for example, for further processing an interpretation) of image data related to a cornea-reflected image, for example, via a camera or other optical array.

In one embodiment, the light source and optical mask are contained within a portable housing. In one embodiment, the light source, optical mask, and at least one processor are contained within a portable housing. In one embodiment, the light source, optical mask, and at least one processor are contained within a desktop, table mounted, or device mounted housing.

In one embodiment, the optical mask comprises a plurality of transparent apertures comprising multiple apices. In one embodiment, the optical mask comprises a plurality of translucent apertures comprising multiple apices. In one embodiment, the optical mask comprises a plurality of transparent apertures comprising multiple edges. In one embodiment, the optical mask comprises a plurality of translucent apertures comprising multiple edges. In one embodiment, the optical mask comprises a plurality of transparent apertures comprising geometries optimized for machine learning ingestion of the captured image. In one embodiment, the optical mask comprises a plurality of translucent apertures comprising geometries optimized for machine learning ingestion of the captured image.

In accordance with one aspect of the disclosed subject matter, for example, a system of corneal (or other anatomical) surface measurement may generally comprise: a light source to transmit incident light through an optical mask, the optical mask comprising a light attenuation portion to impede transmission of the incident light and a plurality of apertures to allow transmission of a selected wavelength and intensity of the incident light; the plurality of apertures defining an image of a pattern that the incident light creates when it is cast on a portion of an anatomy of a patient; an image sensor to capture data representative of the image that is reflected from the anatomy of a patient; and a processing resource to assess a condition of the anatomy based upon the data.

Implementations are disclosed wherein the light source is operative to transmit the incident light in the selected wavelength, wherein ones of the plurality of apertures are transparent in the selected wavelength, and wherein ones of the plurality of apertures are translucent and operative to transmit only light having the selected wavelength.

Systems are disclosed wherein ones of the plurality of apertures comprise a plurality of apices, ones of the plurality of apertures comprise a plurality of edges, or both.

In some implementations, a system may further comprise a light diffusing element interposed between the light source and the optical mask. In some circumstances, it may be desirable that such a light diffusing element transmits the incident light in the selected wavelength.

Additionally or alternatively, a system as describe below may further comprise a lens to collimate the pattern on the image sensor. In some implementations, an image sensor is one of a charge-coupled device and a complementary metal oxide semiconductor sensor, though the following description is not so limited.

In accordance with another aspect of the disclosed subject matter, a method of corneal (or other anatomical) surface measurement may generally comprise: providing a light source to illuminate an area of a patient's anatomy with incident light of a selected wavelength and intensity; interposing an optical mask between the light source and the area; employing the optical mask to cast a pre-determined pattern of the incident light on the area; capturing an image of the pattern using an image sensor to create image data; and interpreting the image data to assess a condition of the area. In some instances, providing a light source may comprise transmitting the incident light in the selected wavelength.

Methods are disclosed wherein employing the optical mask comprises using a plurality of apertures to transmit the incident light in the selected wavelength. In that regard, some methods are disclosed wherein ones of the plurality of apertures are transparent in the selected wavelength, and wherein ones of the plurality of apertures are translucent and operative to transmit only light having the selected wavelength; combinations of the foregoing are contemplated as set forth below.

Some disclosed methods comprise employing a lens interposed between the area and the image sensor, the lens being operative to collimate the image of the pattern on the image sensor. Further methods are disclosed wherein capturing an image comprises using one of a charge-coupled device and a complementary metal oxide semiconductor sensor.

As will be more apparent from a review of the description to follow, in some embodiments, the systems and methods have utility in situations wherein the area (of a patient's anatomy sought to be imaged) is a cornea of an eye.

### BRIEF DESCRIPTION OF THE DRAWINGS

FIG. 1 is a schematic illustration of an example embodiment of a system that enables users to capture corneal surface information for automated analysis.

FIG. 2 is a schematic illustration of an example embodiment of a system with diffusing element and camera that enables users to capture corneal surface information for automated analysis.

FIG. 3 is a schematic illustration of an example embodiment of a system with diffusing element and smartphone that enables users to capture corneal surface information for automated analysis.

FIG. 4 is a schematic illustration of an example embodiment of a system with a partial cylindrical design and camera that enables users to capture corneal surface information for automated analysis.

FIG. 5 is a schematic illustration of an example embodiment of a system with a partial cylindrical design and smartphone that enables users to capture corneal surface information for automated analysis.

FIGS. 6A through 6F are schematic illustrations of an example embodiment of a system that enables users to capture corneal surface information for automated analysis.

FIG. 7 is a cross-sectional schematic illustration of an example embodiment of a system that enables users to capture corneal surface information for automated analysis.

FIG. 8 is an exploded schematic illustration of an example embodiment of a system that enables users to capture corneal surface information for automated analysis.

FIGS. 9A through 9E are exploded schematic illustrations of an optical assembly of an example embodiment of a system that enables users to capture corneal surface information for automated analysis.

FIG. 10 is a cross-sectional schematic illustration of the light path of an example embodiment of a system that enables users to capture corneal surface information for automated analysis.

FIG. 11 is a functional block diagram of a system capable of executing an example process of data production and processing in the case of one or more patients during one or more encounters.

FIG. 12 is a functional block diagram of data production and processing in the case of one or more patients during one or more encounters, with uni-directional and/or bi-directional data flow to an electronic medical record system in accordance with example embodiments; and

FIG. 13 is a flow diagram illustrating aspects of the operational flow of one implementation of a method of corneal surface measurement.

### DETAILED DESCRIPTION

Certain aspects and features of the disclosed subject matter are now described with reference to the drawings, wherein like reference numerals are used to refer to like elements throughout. In the following description, for purposes of explanation, numerous specific details are set forth in order to provide a thorough understanding thereof. It may be evident, however, that the innovative aspects may be practiced without these specific details. In other instances, well-known structures and devices are shown in block diagram form in order to facilitate a description thereof. Specifically, the present disclosure provides for system and method of corneal surface measurement that may generally comprise hardware and software.

As set forth in more detail below, the present disclosure generally addresses acquisition of image data related to the curvature of a body part, such as a cornea of an eye, that are intended or optimized for ingestion by a machine learning or artificial intelligence algorithm, as opposed to being intended or optimized for review by a technician and then later repurposed or further processed for ingestion by such an algorithm.

In some implementations, artificial intelligence may achieve a simplification of interpretation to the point at which a device as described below may be used by a non-clinician; additionally or alternatively, analysis of the clinical image itself may be simplified, even for a trained clinician or other care giver. Conventional tools, primarily corneal topographers, exist that are adept at detecting keratoconus. These devices obtain a reflected image from a patient's cornea and produce a color-coded map to be interpreted by a clinician. The output displayed by such existing tools typically includes a number of calculated parameters that may aid the clinician in making a diagnosis. The aim of these devices is to produce output that is interpretable by a human, who may then make judgments based upon training and experience. To the extent that artificial intelligence (“AI”) has heretofore been employed to analyze such images, the AI has generally been made to ingest (i.e., to load, process, and to examine) the output and parameters that have been primarily produced for human interpretation. In essence, this requires two sets of mathematical computations: the first set is the translation of the reflected image from the cornea into an output that may be interpreted by a human clinician or care provider; the second represents a computation of that reflected image that is produced solely to allow a machine or an algorithm make an inference about the presence of a particular pathology.

In a departure from conventional methodologies, the approach set forth below is to have an AI engine (or algorithm, module, hardware component, software element, etc.) directly ingest the reflected image (i.e., the image reflected from a patient's cornea or other area of a patient's anatomy) without the cumbersome and inefficient intermediate task of producing a human-interpretable intermediate output. The advantages of the proposed approach are twofold: first, by reducing the computational load, the software and hardware may both be made much simpler, cheaper, and more compact; second, the intermediate output created by conventional corneal topography devices for human interpretation requires a great deal of technical training (both from an anatomical standpoint as well as from a device operation standpoint) to have any clinical value—this expertise (in anatomy, electronics, or both) may be minimized or avoided in the instant approach.

Specifically, employment of an AI engine or machine learning technologies may simplify the conventional process by supplying probabilities of the existence of a particular diagnosis in a straightforward manner that may be understood by a user with little to no clinical (or electro-mechanical) training. The present disclosure represents a novel approach to interpretation of ocular surface topography and pathology interpretation, and may be extended to areas of a patient's anatomy beyond the eye.

In that regard, conventional corneal topography devices are designed to facilitate the production of human-interpretable output, but are not optimized for ingestion by AI. As set forth below, one innovation described herein is to create a pattern to be projected onto the cornea, the reflection of which (by the cornea) is more ingestible by artificial intelligence than those currently produced by conventional devices. For instance, the conventional light pattern projected onto the surface of the cornea for diagnostic purposes is a series of concentric rings known as a placido disk. The historical basis for this has to do with the fact that a patient can view concentric rings and make judgments (and provide feedback to a clinician that is relevant to a diagnosis) based upon the patient's perception of the rings' distortion, if any. An inherent limitation in this diagnostic method, however, is that rings lack vertices or apices (corners), while it is features like edges and corners that facilitate computer-assisted image interpretation and analysis. By employing a reflectance mask (or “optical mask”) that is apex-rich, a pattern of incident light falling on a portion of a patient's anatomy (e.g., such as a cornea) may be produced that, while less interpretable to a human being, is more aligned with the sort of images that AI algorithms and machine learning engines require (or prefer) to make inferences or diagnostic suggestions. In some aspects of the present disclosure, such an apex-rich optical mask may present a significant distinction from conventional technologies.

In current ophthalmic treatment regimes, an eye's tear film is typically evaluated by measuring the amount of time that the film is stable before it spontaneously breaks up or decomposes. In the process of selectively projecting an apex-rich pattern of light (e.g., having a desired wavelength and intensity) over a large surface of the cornea, an AI engine or other algorithm may be enabled to distinguish patterns of breakup that may relate to (or provide insight into) a “dry eye” diagnosis.

Additionally, a system and method as set forth herein may be capable of mapping the tear film over a contact lens, and it is believed to be the case that the tear film break up pattern correlates with satisfaction and comfort of a particular patient with a particular contact lens. In this case, a clinician, optometrist, or other care provider may be able to select a compatible contact lens for a particular patient's cornea topography much more rapidly than is currently possible.

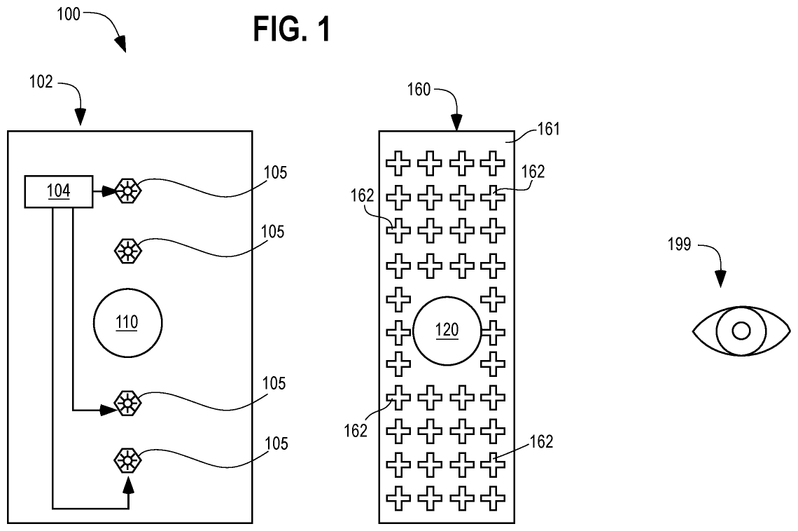

Turning now to the drawing figures, FIG. 1 is a schematic illustration of an example embodiment of a system that enables users to capture corneal surface information for automated analysis. In FIG. 1, the reference numeral 100 refers, in general, to a system enabling capture of corneal (or other anatomical) surface information for automated analysis. In the FIG. 1 implementation, system 100 comprises a substrate 102 having a viewing aperture 110 and at least one light source 104 arranged to transmit light through at least one optical mask 160. In one embodiment, the light source may comprise light-emitting diodes 105. In some implementations, optical mask 160 may comprise a portion to impede or prevent the transmission of light (reference numeral 161) and a plurality of apertures (reference numerals 162) to allow the transmission of light, as is generally known in the art; specifically, various techniques and materials for constructing light attenuation masks or filters such as optical mask 160 are generally known in the art, though none exist that perform the functions in the context set forth herein. The present disclosure is not intended to be limited by any particular material for light attenuation portion 161 or geometry of apertures 162. For example, in one embodiment, apertures 162 may be circular or oval; additionally or alternatively, apertures 162 may be formed to comprise multiple apices (such as the “plus” shape illustrated in FIG. 1, though stars, pinwheels, asterisks, and other shapes are contemplated for apertures 162). In one embodiment, the optical mask 160 comprises a plurality of translucent (rather than transparent) apertures 162; such apertures 162 may comprise multiple apices or edges. Additionally or alternatively, the optical mask 162 may comprise a plurality of transparent (rather than translucent) apertures 162 comprising multiple edges or apices.

As noted above, the optical mask 160 may generally be constructed such that the plurality of apertures 162 comprise geometries that are optimized for machine learning ingestion of an image captured by system 100. In one embodiment, the light source 104 may comprise light-emitting diodes 105, as noted above, though other light sources having utility in medical imaging or ophthalmic applications may also be used. In one embodiment, reference numeral 199 may refer to the cornea of an eye.

In operation, system 100 is operative to direct light from light source 104 through optical mask 160 (specifically, through apertures 162, while other light is attenuated by light attenuation portion 161) to a cornea 199 of an eye. Light reflecting off of cornea 199 is transmitted through viewing aperture 120 in optical mask 160 and through viewing aperture 110 in substrate 102 so as to be captured via a suitable imaging device as set forth in more detail below.

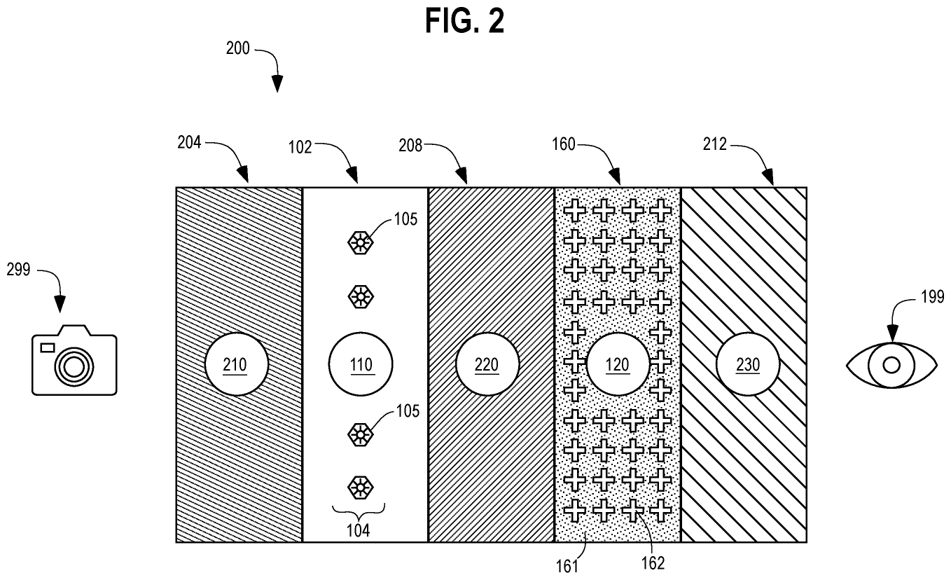

FIG. 2 is a schematic illustration of an example embodiment of a system with diffusing element and camera that enables users to capture corneal surface information for automated analysis. In FIG. 2, system 200 is illustrated as generally comprising at least one light diffusing element 208 and a camera or optical array (reference numeral 299) that enables users to capture image data related to a surface of the cornea 199 of an eye. In one embodiment, reference numeral 299 may refer to a camera, imaging apparatus, or other optical array arranged to record an image reflected from the cornea 199; alternatively, as set forth below, reference numeral 299 may refer to a device, such as a tablet computer, wireless telephone, or other suitable portable device embodied in or comprising such a camera, imaging apparatus, or other optical array. Those of skill in the art will appreciate that references to “imaging device 299” are intended to mean any or all of the foregoing, either independently or in cooperation.

In one embodiment, a substrate 204 is provided with an aperture 210 arranged to align with a camera or visual array associated with or embodied in imaging device 299; in some implementations, this configuration may facilitate alignment of light reflected from cornea 199 such that the light traveling back to imaging device 299 (i.e., from right to left in FIG. 2) is properly registered to impinge on a desired or necessary portion of an image capture hardware element (e.g., such as a charge-coupled device (CCD) or other image sensor array). Light source 104 (which, as noted above, may be embodied in or comprise light-emitting diodes 105 or other point-sources of emitted light) may be disposed on or arranged in connection with substrate 102 as set forth above with reference to FIG. 1; light source 104 may transmit light through at least one optical mask 160 as set forth above and as illustrated in FIG. 2. In the FIG. 2 implementation, system 200 comprises at least one light diffusing element 208 with an aperture 220 arranged to align (as with apertures 110 and 210) with a camera or visual array associated with or embodied in imaging device 299 and an optical mask 160 such as described above. In that regard, optical mask 160 comprises a portion to impede or prevent the transmission of light (light attenuation portion 161) and a portion to allow the transmission of light through a plurality of apertures 162 as the light travels to the cornea 199 (i.e., from left to right in FIG. 2). Aperture 120 may be aligned with the others noted above and below, such that the aperture 120 is arranged to align with operative portions of the imaging device 299 in a desired manner. As noted above in connection with FIG. 1, the optical mask 160 comprises a plurality of apertures 162, which may be of uniform or variable shape and size, may be smoothly curved or comprise multiple apices and/or multiple edges, and may be translucent, transparent, or both.

Without limiting the generalities of the foregoing, it will be appreciated that the optical mask 160 may be implemented to comprise a plurality of apertures 162 having geometries (both individually and collectively) that are favorable to or optimized for machine learning ingestion of the data captured by imaging device 299 related to an image of the cornea 199. Specific sizes, shapes, and geometries of the individual apertures 162, their relative transparency or translucency, and the relative placement of each in an array or field on optical mask 160 may be application-specific, or may vary as a function of the capture and processing capabilities of imaging device 299, the nature of the pathology sought to be detected, overall resolution of the image sought to be captured, manufacturing costs, material composition and other requirements, and other considerations related to construction and desired operational characteristics of optical mask 160, or a combination of these and a variety of other factors.

The FIG. 2 implementation also includes a substrate 212 having an aperture 230 that is also arranged to align (with other apertures 110, 120, 210, and 220) to allow light reflected from the cornea 199 to be transmitted in a desired manner to imaging device 299. In one embodiment, this substrate 212 (that is most distal from imaging device 299) may comprise or be in communication with a mechanical or structural interface that is arranged to contact a portion of a patient's body (e.g., a forehead, cheek bone, orbit, nose bridge, or a combination of these or other reference points) to steady or otherwise to brace system 200 during use.

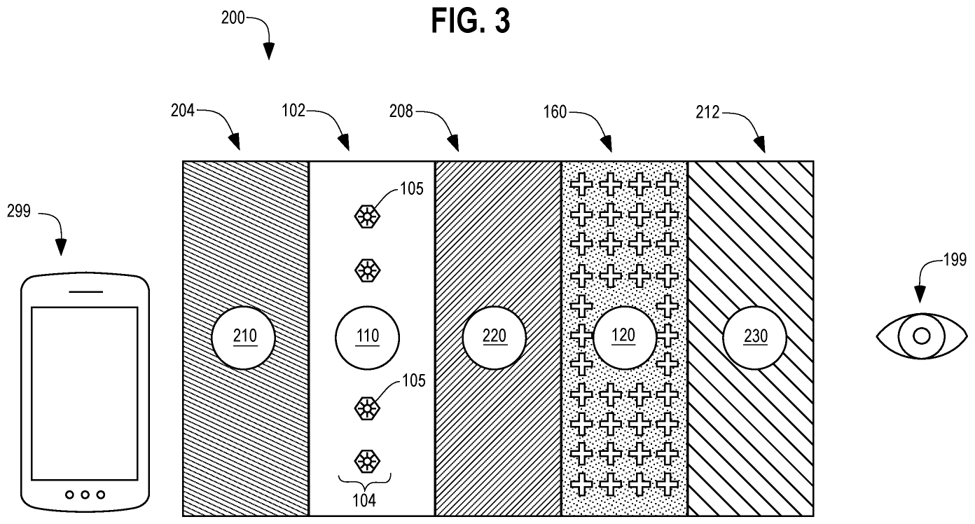

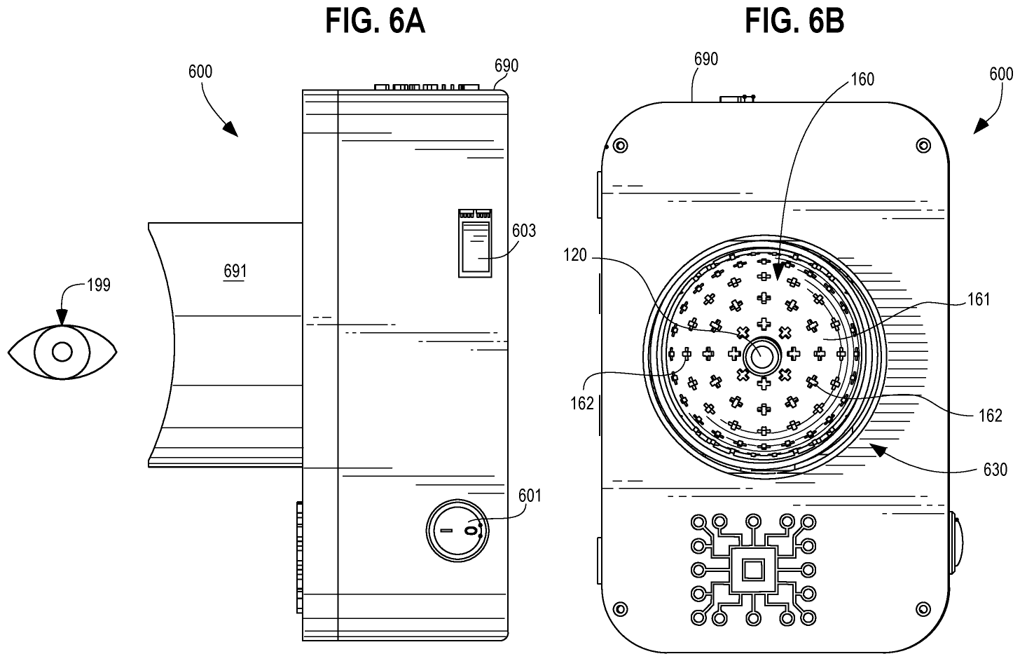

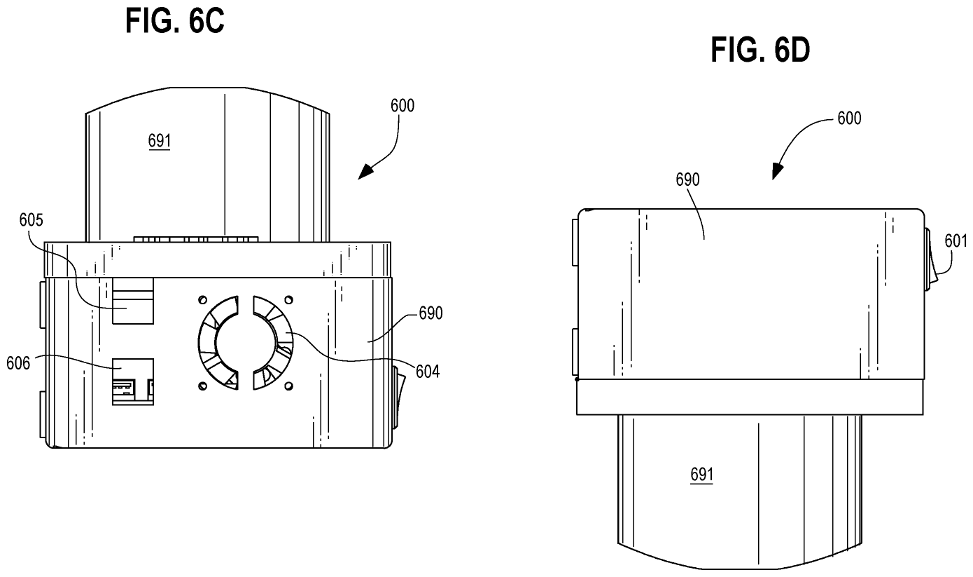

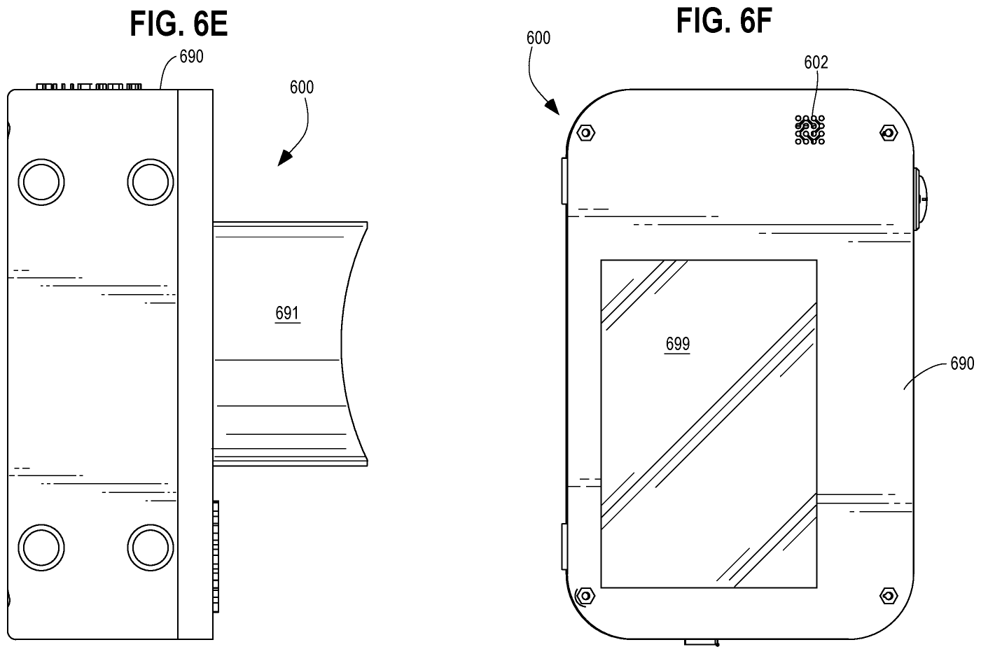

FIG. 3 is a schematic illustration of an example embodiment of a system with diffusing element and smartphone that enables users to capture corneal surface information for automated analysis. In FIG. 3, system 200 is substantially similar to that described above with reference to FIG. 2, but imaging device 299 is depicted as a wireless device (rather than a camera). It is noted that many wireless devices have integrated cameras or other image capture apparatus such as CCD arrays, multi-layer CCD arrays, complementary metal oxide semiconductor (CMOS) sensors, and the like. In that regard, imaging device 299 illustrated in FIG. 3 (and in FIG. 2) may generally be embodied in or comprise a wireless telephone, a tablet or laptop computer, a personal digital assistant (PDA), or any of various other portable devices including or having access to image capture technology as set forth above. In operation (and in some implementations described below with reference to FIGS. 6A through 10), imaging device 299 may be integrated with or attached to system 200 in such a manner as to align apertures 110, 120, 210, 220, and 230 with an operative portion of imaging device 299 such that image data are captured in a desired manner for viewing cornea 199. In that regard, just as substrate 212 may be used to support, secure, steady, or otherwise to brace system 200 against a patient's body, substrate 204 may comprise or be in communication with a mechanical or structural interface that is arranged to contact a portion of imaging device 299 to maintain proper alignment during use such that light reflected from cornea 199 is incident on a desired or required portion of imaging device 299.

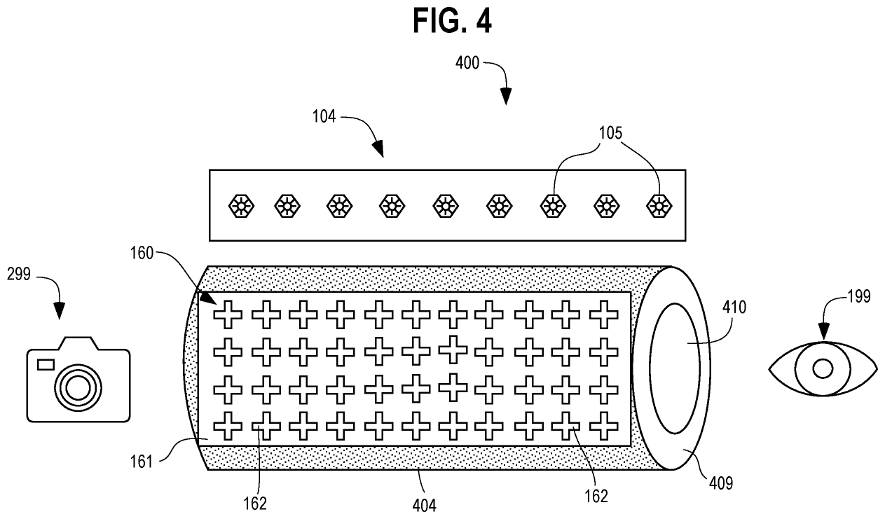

FIG. 4 is a schematic illustration of an example embodiment of a system with a partial cylindrical design and camera that enables users to capture corneal surface information for automated analysis. In FIG. 4, a system 400 is illustrated which generally employs a partial cylindrical and/or partial conical design in connection with the imaging device 299. In the FIG. 4 arrangement, substrate 404 has a partial cylindrical and/or partial conical design and includes a connection to another substrate 409 having an aperture 410 that is arranged to align with a camera or other image capture apparatus at or incorporated into imaging device 299. A least one light source 104 may be arranged to transmit light through at least one optical mask 160 substantially as set forth above, except that the light path is altered based upon the contours of substrate 404. In one embodiment, the substrate 409 includes the aperture 410 configured and operative to align with a camera or visual array at imaging device 299. As with substrate 212, this substrate 409, being most distal from imaging device 299, may comprise or be connected to a mechanical or structural interface arranged to contact a portion of a patient's body such that system 400 may be steadied or properly oriented with reference to a cornea 199 of an eye or other anatomical feature of a patient.

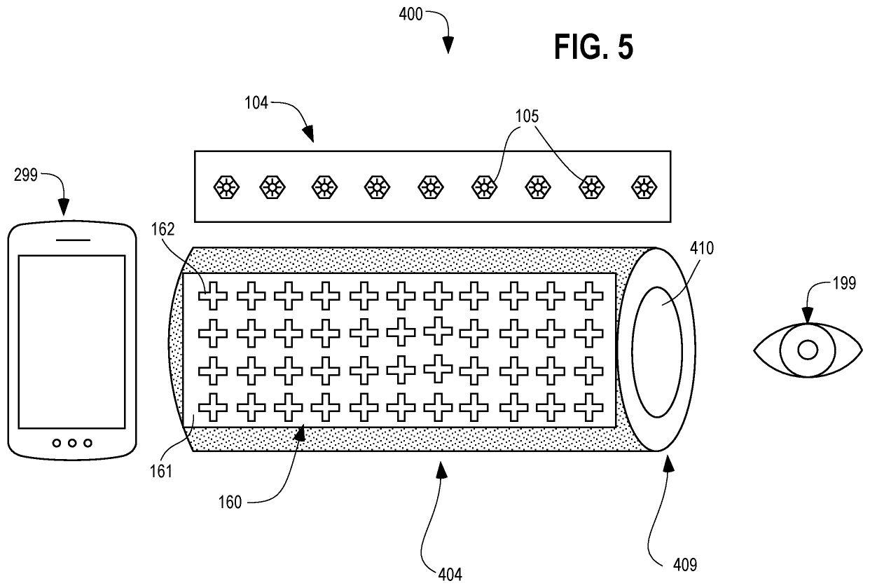

FIG. 5 is a schematic illustration of an example embodiment of a system with a partial cylindrical design and smartphone that enables users to capture corneal surface information for automated analysis. In FIG. 5, system 400 is substantially similar to that described above with reference to FIG. 4, but imaging device 299 is depicted as a wireless device (rather than a camera). As noted above, the present disclosure is not intended to be limited by the specific implementation of the imaging device 299, nor by the form factor, imaging technology, nor degree of integration of same. In that regard, the imaging device 299 depicted in FIGS. 4 and 5 may be integrated with or attached to system 400 in such a manner as to align imaging device 299 such that image data are captured in a desired manner for viewing cornea 199. System 400 may further comprise a mechanical or structural interface (not illustrated in FIGS. 4 and 5 for clarity) that is arranged to contact a portion of, or otherwise physically couple to, imaging device 299 to maintain proper alignment during use such that light reflected from cornea 199 is incident on a desired or required portion of imaging device 299.

FIGS. 6A through 6F are schematic illustrations of an example embodiment of a system that enables users to capture corneal surface information for automated analysis. It is noted that reference numeral 600 refers, in general, to a system such as that described above with reference to FIGS. 1 through 5, but that more physical structure apparent to a user of system 600 is shown (e.g., FIGS. 6A through 6F are less abstract than FIGS. 1 through 5). As depicted in the drawing figures, system 600 may generally comprise a power button or other actuator (reference numeral 601), a power indicator or other indicum that power is currently available or currently being drawn (reference numeral 602), a charge indicator or other indicum of remaining battery power or estimated operational time remaining at a given battery charge level (reference numeral 603), a ventilation aperture (reference numeral 604) to allow for cooling of electronics and power components of system 600 (such as with the assistance of a fan (704) or other cooling mechanism), a charging port (reference numeral 605) to receive external power for charging internal batteries, a data port (reference numeral 606) enabling bi-directional data communications between electronic components associated with system 600 and an external data processing system, an optical assembly (reference numeral 630) for acquiring the image data of a cornea 199 of an eye substantially as set forth herein, and an electronic display screen 699 (that may incorporate touch control in some implementations) to enable a user to view images obtained by system 600, as well as to interface with internal electronics of system 600 and to control operation of same.

The foregoing and other hardware components, electronics, and necessary structural supports or ancillary functional and operational elements (such as electronic data busses, electrical connections, battery supplies, memory interfaces, electronic device controllers, and the like, some of which have been omitted from the drawings for clarity) may be supported or maintained in a housing or enclosure (generally identified at reference numeral 690). In that regard, housing 690 may be constructed of plastics, polymers, acrylics, metals or metal alloys, laminated materials such as fiberglass or carbon composites, or other materials generally known in the art and generally suitable for electronics and component manufacturing methodologies. The present disclosure is not intended to be limited by the nature or structural characteristics of the housing 690 that is used to enclose the operative components of system 600.

Similarly, power buttons (such as 601), power indicators (such as 602), charge indicators (such as 603), ventilation apertures (such as 604), ports (such as charging port 605 and data port 606), and electronic displays (such as 699) are generally known in the art, and are thus not described in more detail here for the sake of brevity. It is noted, however, that the size, placement, and relative orientations of these components (for example, positioning with respect to others of the features, components, buttons, or interface elements) on, in, or in connection with housing 690 are generally design choices and may be application-specific as a function of the overall desired operational characteristics or functionality of system 600, manufacturing costs, desired user interface (UI) or user experience (UX) requirements, specifications, or parameters, or a combination of these and a variety of other factors. For example, where display 699 is not a “touch-screen” or “touch-active” display, accommodations may be made for interface mechanisms (buttons, rocker panels, two-dimensional switches, and the like) to be disposed somewhere on housing 690 to enable a user to access and interact with the operational characteristics of system 600.

On the other hand, optical assembly (reference numeral 630) for acquiring the image data of a cornea 199 of an eye represents a departure from conventional technologies, and enables novel imaging and data capture. In that regard, housing 690 and the other components of system 600 (and, indeed, of systems 100, 200, and 400) are intended to be so sized, structured, positioned, and interconnected as to support operation of optical assembly 630 in any and all of the several implementations set forth herein. It will be appreciated that any of various alternatives may be employed for any given element of system 600, provided however, that optical assembly 630 is enabled to acquire image data of the cornea 199 substantially as illustrated and described.

In some implementations, housing 690 may include a distal portion 691; it will be appreciated that, in this context, “distal” refers to that portion of housing 690 that is furthest from an imaging device 299 (not shown in FIGS. 6A through 6F) and closest to the cornea 199 of a patient's eye (or other anatomical feature to be imaged). Distal portion 691 may comprise, or may be integral with, for example, substrate 212 described above with reference to FIGS. 2 and 3; in use, distal portion 691 may be placed against a portion of a patient's body (e.g., a forehead, nose, orbit, cheek, etc.) to support, secure, brace, or otherwise to stabilize system 600 during use.

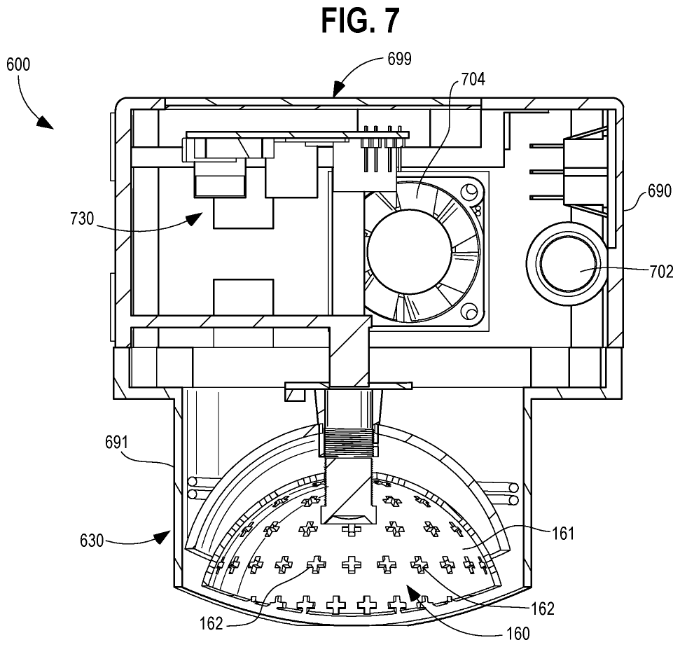

FIG. 7 is a cross-sectional schematic illustration of an example embodiment of a system that enables users to capture corneal surface information for automated analysis, and in that regard, represents the system 600 described above in connection with FIGS. 6A through 6F. Accordingly, system 600 in FIG. 7 is depicted as comprising a display screen 699, a rechargeable battery (reference numeral 702), and a cooling fan 704, which may work in cooperation with ventilation aperture 604, as noted above. In operation, control of system 600 may generally reside at a processing resource (depicted generally in FIG. 7 at reference numeral 730).

Those of skill in the art will appreciate that processing resource 730 may generally comprise one or more microprocessors, field-programmable gate arrays (FPGAs), application specific integrated circuits (ASICs), programmable logic blocks, microcontrollers, or other digital processing apparatus suitable for image acquisition and data processing in accordance with requirements or design specifications of system 600. Typically, processing resource 730 may cooperate with or operate in connection with a memory structure or database (not shown in FIG. 7), which may generally comprise or have access to, by way of example, volatile memory such as random access memory (RAM) in any of its various forms, for instance, static RAM (SRAM), dynamic RAM (DRAM), double-data rate (DDR) RAM, and the like; in some applications, DDR4 RAM may be used as or in connection with processing resource 730. Additionally or alternatively, processing resource 730 may include, have access to, or generally comprise a mass data storage component, such as a non-volatile data storage device, one example of which is an Electronically Erasable Programmable Read Only Memory (EEPROM) store.

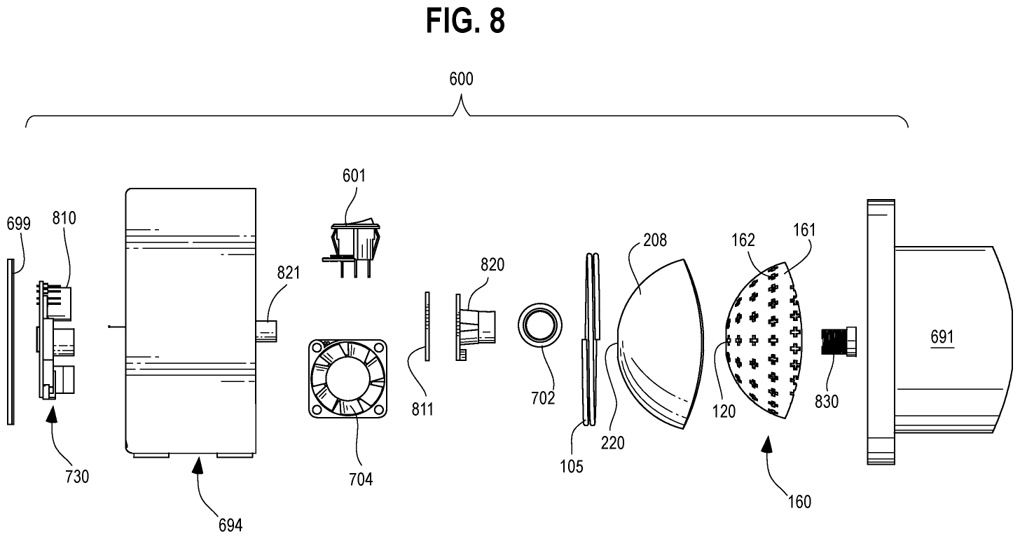

FIG. 8 is an exploded schematic illustration of an example embodiment of a system that enables users to capture corneal surface information for automated analysis. In the depiction of FIG. 8, system 600 may generally include, from left to right, an electronic display 699, processing resource 730, electronic charge controlling circuitry 810 (which may include or be operably coupled to charging port 605), a rear panel or enclosure (reference numeral 694) associated with or integral with housing 690, a positioning post (reference numeral 821) to facilitate support, positioning, or spatial registration of an image sensor (820, noted below), a power switch or button 601, a cooling fan 704, an electronic breadboard 811 (e.g., to support operation of a image sensor 820 in cooperation with processing resource 730), an image sensor 820, at least one rechargeable battery 702, a light emitting diode strip 105 (e.g., in a helical pattern or other suitable arrangement), a translucent light diffusing element 208, an optical mask 160, a macrophotography lens 830, and a distal portion 691 of housing 690 that is adapted to abut against a portion of a patient's body as described above.

It will be appreciated that image sensor 820 may be embodied in or comprise a CCD array, a multi-layer CCD array, a CMOS sensor, or a combination of these and other image sensing technologies; image sensor 820 may be referred to as a “camera” in some instances, but is not intended to be so limited. Similarly, macrophotography lens 830 may be embodied in or comprise any of myriad lenses or focusing or collimating assemblies operative to focus light reflected from a cornea 199 of an eye onto image sensor 820 in a desired manner. In that regard, the collimating qualities, focusing aspects, functional requirements, or other operational characteristics of macrophotography lens 830 may be application-specific in some instances, or may be selected as a function of cost, reliability, optical clarity or resolution, or as a combination of these and a variety of other factors. The present disclosure is not intended to be limited by the nature, functionality, or operational parameters of macrophotography lens 830 or image sensor 820.

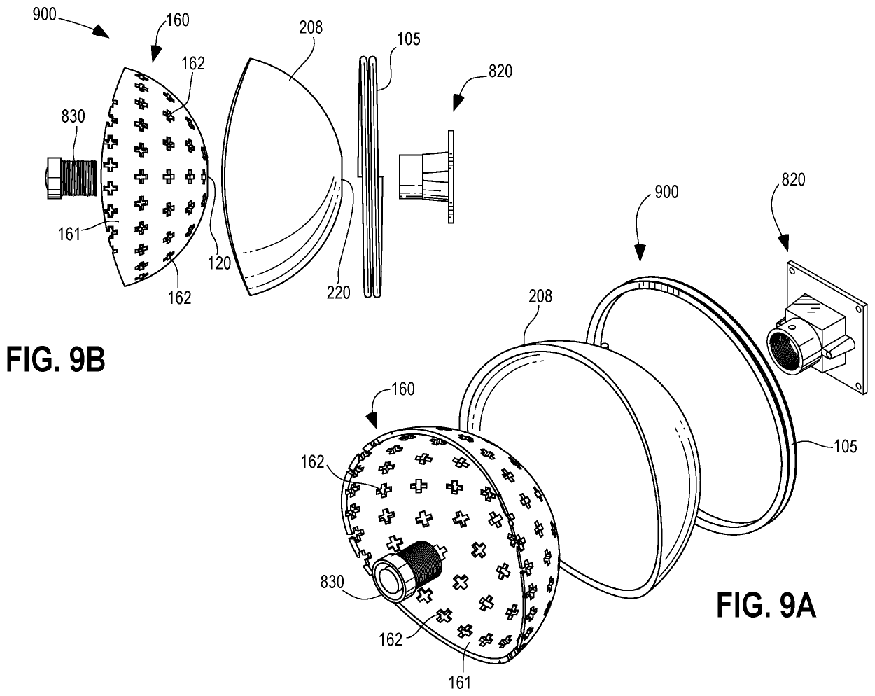

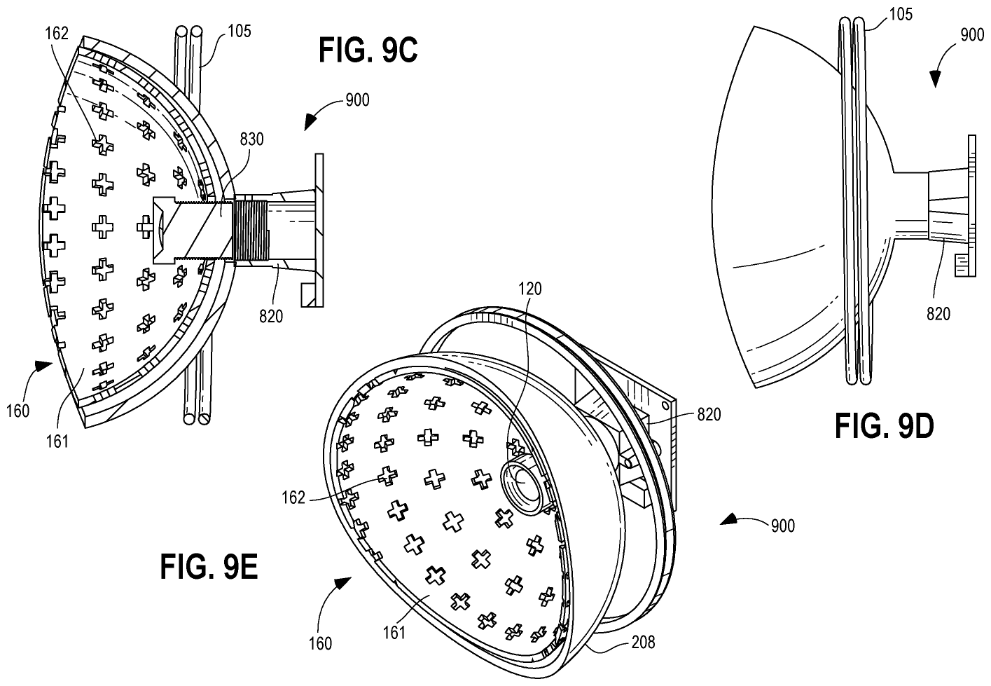

FIGS. 9A through 9E are exploded schematic illustrations of an optical assembly of an example embodiment of a system that enables users to capture corneal surface information for automated analysis. In FIGS. 9A through 9E, the reference numeral 900 generally refers to such an optical assembly to be incorporated into a system such as described above with reference to FIGS. 1 through 8. In the illustrated implementation, optical assembly 900 comprises macrophotography lens 830, optical mask 160, light diffusing element 208, a light source 104 (such as a light emitting diode 105, which is illustrated in FIGS. 9A through 9E as a light emitting diode strip arranged in a helical or circular pattern), and image sensor 820.

In operation, these components of optical assembly 900 are to generate light at light source 104 (e.g., at a desired wavelength and intensity via light emitting diode 105 or some other suitable source, depending upon the nature of the light to be produced) which is diffused via light diffusing element 208 and cast upon optical mask 160. Optical mask 160, in turn, allows a cornea 199 of an eye to be illuminated via light transmitted through apertures 162 (which, as noted above, may be translucent or transparent, e.g., as a design choice or as an application-specific requirement, for instance, as a function of a specific ophthalmic pathology that is to be detected). In that regard, light that is incident on a cornea 199 may reflect back to macrophotography lens 830, which focuses the reflected light and transmits same (e.g., via apertures 120 and 220) to image sensor 820 for processing. This functionality and the interconnection of the various components are best illustrated in FIGS. 9A and 9B. By way of additional detail, FIG. 9D is a profile view and FIG. 9E is a three-quarters perspective view of this same optical apparatus 900.

FIG. 9C is a cross-sectional view of this same optical apparatus 900 illustrated in FIGS. 9A and 9B, and depicts macrophotography lens 830 threadably engaged with image sensor 820 (e.g., through apertures 120 and 220 shown in FIGS. 1 through 3). It will be appreciated that other engagements are contemplated, and that press-fit, friction-fit, “slot and tab” or “tongue and groove” arrangements, adhesives, welds, and other attachment structures or methodologies may be employed to engage macrophotography lens 830 and image sensor 820 (e.g., either as a design choice or as a function of the specific structural or operational characteristics of the components).

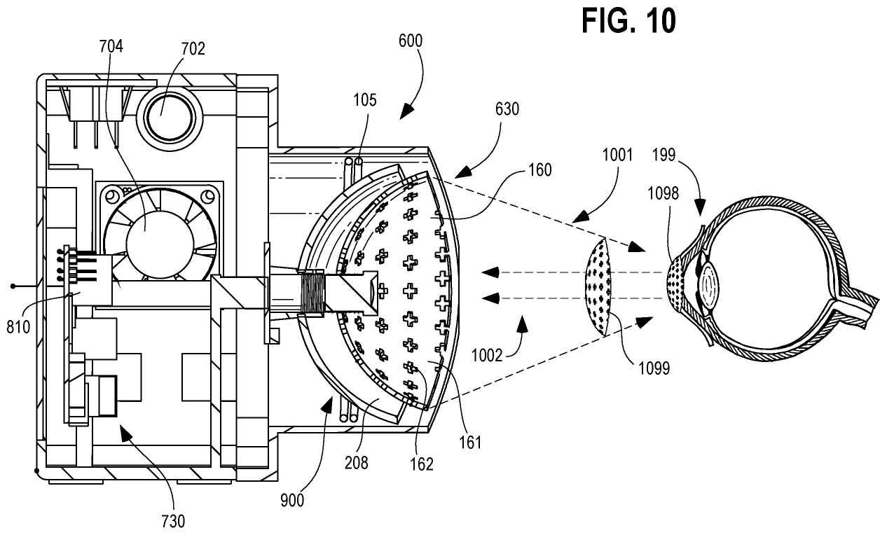

FIG. 10 is a cross-sectional schematic illustration of the light path of an example embodiment of a system that enables users to capture corneal surface information for automated analysis. In FIG. 10, system 600 is depicted in operation (though, as noted above, other implementations of system 600 are contemplated).

It will be appreciated that, in FIG. 10, reference numeral 1001 schematically depicts, via the dashed arrows from left to right, a light path of “outgoing” rays (i.e., light of a desired frequency, intensity, and wavelength, emanating from a light source 104 such as light emitting diode 105), passing through a light diffusing element 208, passing through apertures 162 of an optical mask 160, and propagating toward a patient (e.g., toward the cornea 199 of an eye); this is referred to herein as “incident light,” as it is intended to be incident on a portion of the anatomy of a patient, such as cornea 199. Conversely, reference numeral 1002 schematically depicts, via the dashed arrows from right to left, a light path of “incoming” rays or “reflected” light (i.e., light that has reflected off of the cornea 199) that represent an image 1099 of a pattern that incident light 1001 made when it struck the cornea 199 (see reference numeral 1098). It will be appreciated that incoming rays 1002 may be focused or otherwise collimated by macrophotography lens 830 for suitable receipt by image sensor 820 and subsequent processing (e.g., by image sensor 820, processing resource 730, or both).

It is noted that a cornea 199 of an eye has been mentioned above, but other tissues, structures, or other surfaces of a patient (and other optical targets) are contemplated for operation of the disclosed systems and methods. While distal portion 691 of housing 690 is illustrated and described in connection with engaging anatomical structures near a human eye, the present disclosure is not intended to be so limited.

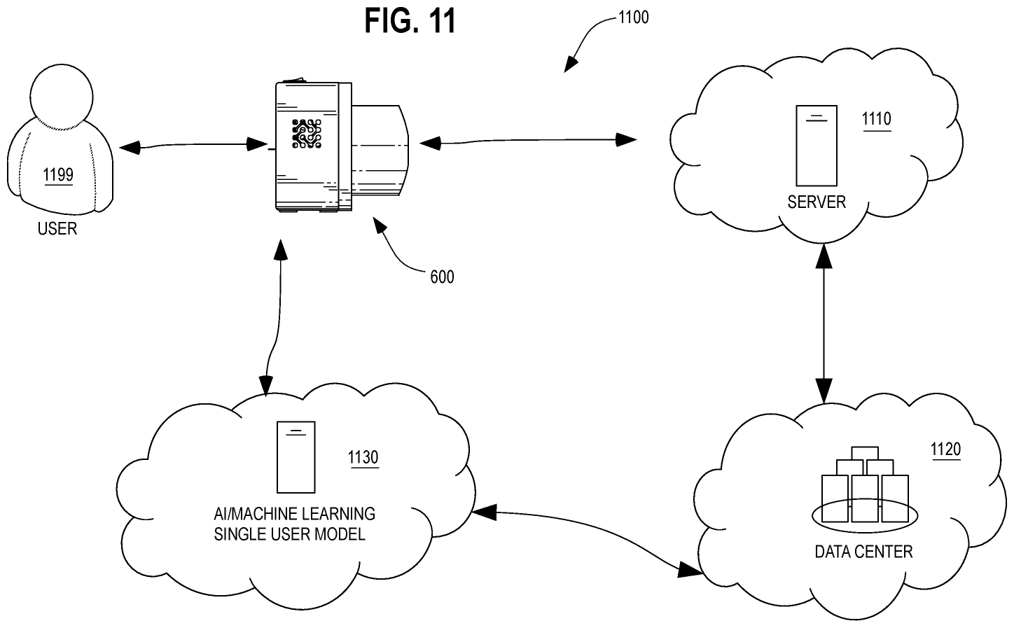

FIG. 11 is a functional block diagram of a system capable of executing an example process of data production and processing in the case of one or more patients during one or more encounters. In FIG. 11, one system architecture 1100 is illustrated that may have utility in connection with data capture and processing for one or more patients, and with storing such data in computer memory. In the FIG. 11 arrangement, reference numeral 600 generally refers to an apparatus, device, system, or other hardware element or combination of components configured and operative to acquire or otherwise to receive data; examples of such a system 600 are described in detail above with reference to FIGS. 1 through 10. In some implementations described above, these data may comprise images of patient tissue (e.g., such as image 1099 of a patient's cornea 199), though other types of image data are contemplated, for instance, as a function of the area of a patient's anatomy to be imaged and the nature of a pathology sought to be detected (for instance, acne, melanoma, eczema, or other dermatological pathologies).

As noted above, system 600 may be configured and operative to receive input, feedback, operational parameters, and other instruction sets or commands from a user 1199 (e.g., via a touch screen display 699 or other user interface mechanism); additionally or alternatively, such a system 600 may be configured and operative to receive such instruction sets or commands from networked or remote compute resources such as server 1110 and artificial intelligence (AI) engine 1130 as set forth below. The bi-directional data communications pathways illustrated by the double arrows in FIG. 11 may be wired or wireless, as is generally known in the art.

In operation, user 1199 of a system 600 that enables capture of corneal surface information for automated analysis may engage an interface provided at system 600 (such as via any of various and common user interface technologies, including without limitation, touch screen display 699) to initiate, terminate, or otherwise control capture of image data from an area of a patient's anatomy substantially as set forth above with reference to FIGS. 1 through 10. In the context of FIG. 11, it will be appreciated that system 600 generally represents at least one computing device configured and operative to capture and process an image of an anatomical feature substantially as set forth herein, but that other such devices, apparatus, systems, and arrangements of components may be similarly suited to effectuate similar functionality.

In some implementations, data captured or received by, or otherwise transferred or input to, system 600 may comprise the name, address, license number, or other identifying information associated with the user 1199 of system 600, the name or corporate identification number of an entity employing or affiliated with the user 1199, the name, address, telephone number, credit card information, and insurance parameters for a patient who is the subject of the image data being acquired by system 600, and the like.

In accordance with some aspects of the present disclosure, system 600 in general, and processing resource 730, in particular, may be configured and operative (e.g., in cooperation with memory resources that are not illustrated in the drawing figures for clarity) to perform statistical and/or machine learning and and/or artificial intelligence analysis of captured images (such as image 1099, for instance). Such computing may be local (e.g., entirely resident on system 600, facilitated by processing resource 730), remote (e.g., executed at server 1110 or 1130, in cooperation with data on servers at data center 1120), or a combination of both. In any event, data representative of results or conclusions based upon such statistical analysis may be presented at system 600 (e.g., on a display screen 699), transmitted to or retained at server 1110 for subsequent analysis, transmission for display, or both, transmitted to AI engine 1130 to be used as seed data for further AI training, or a combination of any or all of the foregoing.

In this context, the server depicted at the upper right of FIG. 11 (reference numeral 1110) represents at least one computing device, computer server, processing resource, or remote electronic device that is configured and operative to receive data and, optionally, analysis or interim analytical results, from a system that enables users to capture corneal surface information for automated analysis (i.e., system 600). Similarly, data center (reference numeral 1120) generally represents a computing device, computer server, processing resource, or remote electronic device that is configured and operative to receive patient information and images from system 600, server 1110, or both, and AI engine (reference numeral 1130) may be embodied in or comprise a computing device, computer server, processing resource, or remote electronic device that is configured and operative to perform statistical, machine learning, and/or artificial intelligence analysis of accumulated data acquired or otherwise transmitted from system 600, data center 1120, or both.

As noted above, it will be appreciated that server 1110, data center 1120, and AI engine 1130 may generally be embodied in or comprise a computer server, a desktop or workstation computer, a laptop or portable computer or tablet, or a combination of one or more of such components. In operation, these devices (individually or in cooperation) may be employed to initiate, instantiate, or otherwise to effectuate data processing operations as is generally known in the art. In that regard, these components 1110, 1120, and 1130 may be embodied in or include one or more physical or virtual microprocessors, FPGAs, application specific integrated circuits (ASICs), programmable logic blocks, microcontrollers, or other digital processing apparatus, along with attendant memory, controllers, firmware, and network interface hardware (not illustrated in FIG. 11 for clarity), and the like that facilitate remote data processing or support of operations executed by system 600. For example, components 1110, 1120, and 1130 may generally comprise multiprocessor systems, microprocessor-based or programmable consumer electronics, personal computers (“PCs”), networked PCs, minicomputers, mainframe computers, and similar or comparable apparatus for general purpose or application-specific data processing. Various implementations of components 1110, 1120, and 1130 may be deployed in distributed computing environments in accordance with which tasks or program modules may be performed or executed by remote processing devices, which may be linked through a communications network; alternatively, some of the functionality of components 1110, 1120, and 1130 may be combined into a single compute or processing resource as is generally known. Those of skill in the art will appreciate that any of various computer servers, work stations, or other processing hardware components or systems of components may be suitable for implementation in the configuration illustrated in FIG. 11, and that the disclosed subject matter is not limited to any particular hardware implementation or system architecture employed at any of the various components 1110, 1120, or 1130.

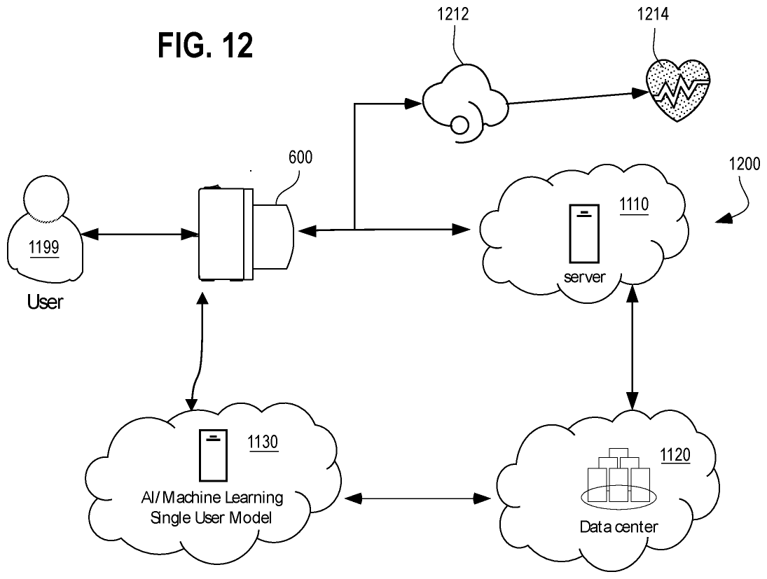

FIG. 12 is a functional block diagram of data production and processing in the case of one or more patients during one or more encounters, with uni-directional and/or bi-directional data flow to an electronic medical record system in accordance with example embodiments. In FIG. 12, there is illustrated a system architecture 1200 for data capture and processing of one or more patients and for storing acquired data in computer memory (e.g., at data center 1120, server 1110, AI engine 1130, system 600, or a combination of the foregoing). In that regard, the system architecture 1200 in FIG. 12 is generally substantially similar to that illustrated in FIG. 11 and described above (see reference numeral 1100). One notable difference, however, is that system architecture 1200 accommodates an application programming interface (API, reference numeral 1212) and a medical data management system (reference numeral 1214), which may be owned, leased, maintained, or operated by, or otherwise under the control of, a medical practice, facility, hospital, clinic, government agency, or other third party, for example, or it may be owned or operated by the entity that controls system architecture 1200.

In operation, API 1212 may generally enable bi-directional data communication between system 600 (or other elements of system architecture 1200) and medical data management system 1214 (which, as noted above, may be controlled by a third party). In some implementations, such a patient medical data management system 1214 may be an electronic medical record system or an electronic health record system such as may be maintained by a county, state, or federal medical board, for example, or by a private research facility, university, medical practice, or for-profit life sciences research entity.

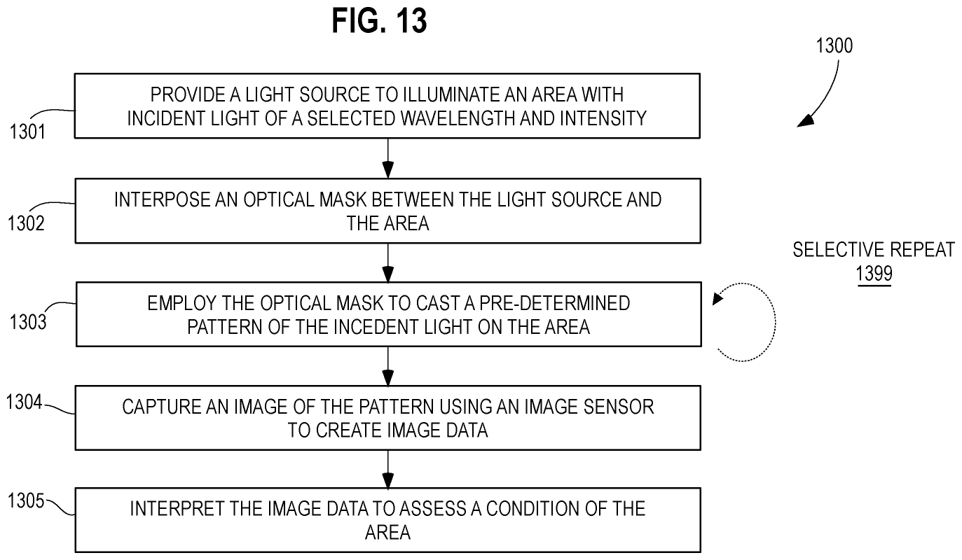

FIG. 13 is a flow diagram illustrating aspects of the operational flow of one implementation of a method of corneal surface measurement. As indicated in FIG. 13, such a method 1300 may begin by providing a light source to illuminate an area of a patient's anatomy with incident light of a selected wavelength and intensity (block 1301). As set forth above, a light source (such as reference numeral 104) employed for this purpose may be embodied in or comprise a light emitting diode 105 or other source of electromagnetic radiation, such as an incandescent light bulb or array of bulbs. The wavelength and intensity of the light (i.e., electromagnetic radiation) applied to the area may be application-specific, for instance, or it may be selected as a function of type of pathology sought to be detected, the nature of the light source 104, or a combination of these and other factors.

Method 1300 may continue by interposing an optical mask between the light source and the area to be illuminated, as indicated at block 1302. An optical mask, such as described above in connection with reference numerals 160, 161, and 162, may be manufactured of aluminum, stainless steel, nickel, or other suitable metals, for example; additionally or alternatively, some portions (or the entirety) of the structure of an optical mask may be constructed of ceramics, plastics, acrylics, or laminated materials such as fiberglass or carbon composites. In some implementations, apertures (such as described above in connection with reference numeral 162) may simply be holes or voids (i.e., spaces in optical mask 160 where there is no material at all); in other implementations, however, such apertures may include translucent materials (e.g., acrylics, glasses, plastics, etc.) that selectively allow transmission of incident light of a particular wavelength or intensity.

In the illustrated implementation, method 1300 may continue by employing the optical mask to cast a pre-determined pattern of the incident light on the area (block 1303); this operation may be selectively repeated as indicated by the dashed loop at reference numeral 1399. As noted above, this particular operation represents a departure from conventional methodologies which do not contemplate casting complex patterns of incident light on an area of a patient's anatomy (particularly a cornea 199 of an eye).

An image of the pattern of incident light (that is cast on the area of patient's anatomy) may be captured using an image sensor to create image data, as is indicated at block 1304. Many suitable image sensors (such as image sensor 820 described above) are known in the art to have sufficient functionality for this purpose; the present disclosure is not intended to be limited by the nature, functional characteristics, or specific technology employed by any particular image sensor employed to execute the functionality identified at block 1304.

Acquired image data may be interpreted to assess a condition (e.g., determine a pathology) of the area illuminated by the incident light, as indicated at block 1305. As noted above, conditions such as “dry eye” and keratoconus may be detected by the method of FIG. 13, and it may be possible to prescribe appropriate contact lenses or other ophthalmic prosthetics based upon the data captured and interpreted (e.g., at blocks 1304 and 1305).

It is noted that the arrangement of the blocks and the order of operations depicted in FIG. 13 are not intended to exclude other alternatives or options. For example, the operations depicted at blocks 1301 and 1302 may be reversed in order, or they may be made to occur substantially simultaneously in some implementations. Further, one or more of these operations may occur substantially simultaneously with the operations depicted at blocks 1303 and 1304 in instances where it may be desirable to do so, e.g., for efficiency, where processing resources are sufficient, and the like. The capture and interpretation operations at blocks 1304 and 1305 may be combined, for instance, or made to occur substantially concomitantly, in situations in which relevant computer hardware and software (implemented at system 600 and server 1110, for example) have sufficient capabilities. Those of skill in the art will appreciate that the foregoing subject matter is susceptible of various design choices that may influence the order or arrangement of the operations depicted in FIG. 13.

Several features and aspects of a system and method have been illustrated and described in detail with reference to particular embodiments by way of example only, and not by way of limitation. Those of skill in the art will appreciate that alternative implementations and various modifications to the disclosed subject matter are within the scope and contemplation of the present disclosure. Therefore, it is intended that the present disclosure be considered as limited only by the scope of the appended claims.

What is claimed is:

1. A system comprising:
a light source to transmit incident light through an optical mask, the optical mask comprising a light attenuation portion to impede transmission of the incident light and a plurality of apertures to allow transmission of a selected wavelength and intensity of the incident light; the plurality of apertures defining an image of a pattern that the incident light creates when it is cast on a portion of an anatomy of a patient;
an image sensor to capture data representative of the image that is reflected from the anatomy of a patient; and
a processing resource to assess a condition of the anatomy based upon the data.

2. The system of claim 1, wherein the light source is operative to transmit the incident light in the selected wavelength.

3. The system of claim 2, wherein ones of the plurality of apertures are transparent in the selected wavelength.

4. The system of claim 1, wherein ones of the plurality of apertures are translucent and operative to transmit only light having the selected wavelength.

5. The system of claim 1, wherein ones of the plurality of apertures comprise a plurality of apices.

6. The system of claim 5, wherein ones of the plurality of apertures comprise a plurality of edges.

7. The system of claim 1 further comprising a light diffusing element interposed between the light source and the optical mask.

8. The system of claim 7, wherein the light diffusing element transmits the incident light in the selected wavelength.

9. The system of claim 1 further comprising a lens to collimate the pattern on the image sensor. The system of claim 1, wherein the image sensor is one of a charge-coupled device and a complementary metal oxide semiconductor sensor.

11. A method comprising
providing a light source to illuminate an area of a patient's anatomy with incident light of a selected wavelength and intensity;
interposing an optical mask between the light source and the area;
employing the optical mask to cast a pre-determined pattern of the incident light on the area;
capturing an image of the pattern using an image sensor to create image data; and
interpreting the image data to assess a condition of the area.

12. The method of claim 11, wherein the providing a light source comprises transmitting the incident light in the selected wavelength.

13. The method of claim 11, wherein the employing the optical mask comprises using a plurality of apertures to transmit the incident light in the selected wavelength.

14. The method of claim 13, wherein ones of the plurality of apertures are transparent in the selected wavelength.

15. The method of claim 13, wherein ones of the plurality of apertures are translucent and operative to transmit only light having the selected wavelength.

16. The method of claim 11 further comprising employing a lens interposed between the area and the image sensor, the lens operative to collimate the image of the pattern on the image sensor.

17. The system of claim 16, wherein the capturing an image comprises using one of a charge-coupled device and a complementary metal oxide semiconductor sensor.

18. The method of claim 11, wherein the area is a cornea of an eye.
